(19) United States
(12) Patent Application Publication
(10) Pub. No.: US 2007/0059201 A1
(43) Pub. Date: Mar. 15, 2007

(54) DRY ICE PRODUCT CONTAINING ANTIMICROBIAL FORMULATION PREPARED USING CARRIER CHEMICALS

(76) Inventors: Meenakshi Sundaram, Burr Ridge, IL (US); Pasi Ihatsu, Kontiolahti as. (FI)

Correspondence Address: Ms. Linda K. Russell, Air Liquide Intellectual Property Dept., 2700 Post Oak Blvd., Suite 1800, Houston, TX, 77056, US

(21) Appl. No.: 11/457,602

(22) Filed: Jul. 14, 2006

Related U.S. Application Data
(60) Provisional application No. 60/717,518, filed on Sep. 15, 2005.
(60) Provisional application No. 60/717,564, filed on Sep. 15, 2005.

(51) Int. Cl. A61L 2/18

(52) U.S. Cl. 422/28

(57) ABSTRACT

Embodiments of the invention generally provide a treating agent comprising an antimicrobial carrier chemical and a cooling agent to chill and sanitize food products during processing steps, wherein the antimicrobial carrier chemical is in liquid or solid form.

### CROSS-REFERENCE TO RELATED APPLICATIONS

This application claims the benefit under 35 U.S.C. § 119(e) to provisional application No. 60/717,518, filed Sep. 15, 2005, the entire contents of which are incorporated herein by reference.

### BACKGROUND

1. Field of the Invention

The invention relates to the improvement of sanitization techniques used during the processing of food products, more specifically to a treating agent comprising an antimicrobial carrier chemical and a cooling agent to chill and sanitize food products during processing steps.

2. Description of the Related Art

Food safety problems not only originate in the food product itself (e.g., raw ingredients), but also from the environment surrounding the food product. A food product is susceptible to microbial contamination during the processing steps and after the terminal heating process. For example, food can be damaged by microbes, spores, insects, and other sources. Each year economic losses of food and labor due to damage from such sources are more than $100 billion. Currently, food items are preserved using a variety of methods, including refrigeration, fumigation with toxic chemicals, irradiation, biological control, heat exposure, and controlled atmospheric storage.

According to at least one estimate, post/cross contamination from either environment or food contact surfaces is implicated in up to 30% of food poisoning cases. Post/cross contamination also increases the microbial load in finished products, shortening shelf-life and becoming a visual deterrent of quality. As such, the finished product can serve as a carrier of cross-contamination leading to economic losses, as well as health and survival issues involving consumers. For example, Listeria spp is an environmental air-borne pathogen causing listeriosis that can contaminate food products during processing. According to the Center for Disease Control, there were 1850 cases of listeriosis in 1998, including 435 deaths from this disease. Effective methods using sanitizers/disinfectants are crucial to minimize and prevent microbial contamination of foods.

Effective sanitation of food or other items depends on the combination of what is to be sanitized and the sanitation process type. Not all of the currently available technologies can deliver an effective reduction of microorganisms and at the same time prevent product or environmental degradation. It is well known in the art to cool products, such as foods, during processing with some type of refrigerant to slow down the growth of unwanted microbes and enzymatic reactions in foods. For instance, the shelf life and quality of food products are improved by processing, transporting, and storing under refrigerated conditions.

Cooling agents such as solid carbon dioxide (dry ice) or nitrogen are liquid or solid agents that can be used as expendable refrigerants. Water ice is a traditional expendable refrigerant, but has the disadvantage of converting to water after the ice melts. Some solid cooling agents convert from a solid directly to a gas in the process known as sublimation. For example, dry ice sublimes by going directly from a solid to a gas without passing through the liquid stage. The cold temperature of dry ice and the fact that it leaves no residue like water ice makes it an excellent refrigerant in some applications. Food products that must remain frozen can be packed with dry ice during transportation. The contents will be frozen when they reach their destination and there will be no messy liquid residue as found with traditional water ice. In food processing applications, liquids, such as nitrogen, are used to cool and inert the atmosphere during food processing or storage. Liquid cooling agents may also be used for other cryogenic applications, such as the preservation of human tissues and organic bodies.

While refrigeration can retard microbial growth, such treatment does not necessarily kill bacteria. Accordingly, microorganisms can still survive through refrigeration, and worse, some microorganisms can still grow and produce harmful substances during refrigerated storage. Furthermore, it is possible that the refrigerant used to cool a target item or food product can itself be contaminated with pathogenic microorganisms, thus contaminating the target item or food product.

Antimicrobial agents are used to sanitize equipment, provide antiseptic environments, treat water, and sanitize foods. The reaction of antimicrobial agents with microbial cell structures is often irreversible, causing the cells to either become attenuated or die.

One antimicrobial agent that can be used for industrial applications is ozone. However, ozone is very unstable and therefore must be produced at the location of consumption. Production of ozone requires specialized equipment and involves safety issues due to handling of the equipment and feedstock, such as pure oxygen. After the ozone is produced, it must be delivered in some form to the target item as a sanitizer. Ozone is often dissolved or absorbed in water as a mechanism to deliver the unstable ozone to a target item. However, ozone has poor solubility in water. Mixtures of ozone and water typically contain less than about 20 ppm by weight ozone. As a result, large quantities of water relative to the ozone are required if water is used as a delivery agent. Because of the large quantities of water required, the ozone and water cannot be pre-mixed and transported to site. Thus, ozone and water must be mixed on site. Furthermore, when incorporating ozone in wet ice for cooling applications, a large bulk of water is left behind when the wet ice melts.

Therefore, there remains a need to provide antimicrobial properties to cooling agents by incorporating antimicrobial carrier chemicals during processing.

### SUMMARY

Embodiments of the invention generally provide exposing a target item to a treating agent, wherein the treating agent comprises a liquid antimicrobial carrier chemical and a cooling agent. In one embodiment, the invention provides a method of processing a target item, comprising exposing a target item to a treating agent, wherein the treating agent comprises an antimicrobial carrier chemical suspended in a cooling agent, the antimicrobial carrier chemical being in liquid or solid form, and contacting the target item with the antimicrobial carrier chemical.

In another embodiment, the invention provides a method of packaging a target item comprising placing a target item into a container, adding a treating agent to the container, wherein the treating agent comprises an antimicrobial carrier chemical suspended in a cooling agent, the antimicrobial carrier chemical being in liquid or solid form, and contacting the target item with the antimicrobial carrier chemical.

In another embodiment, the invention provides a treating agent comprising an antimicrobial carrier chemical suspended in a cooling agent, wherein the antimicrobial carrier chemical is in liquid or solid form.

### BRIEF DESCRIPTION OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 exhibits the main processing steps entailed by the embodiments of the invention;

FIG. 2 is a schematic illustration of one embodiment of the invention for incorporating an antimicrobial chemical into dry ice and forming pressed blocks of dry ice;

FIG. 3 is a schematic illustration of another embodiment for forming extruded pellets of dry ice containing an antimicrobial chemical; and

FIG. 4 is a schematic illustration of on packaged product embodiment of the invention.

### DESCRIPTION OF PREFERRED EMBODIMENTS

The words and phrases used herein should be given their ordinary and customary meaning in the art by one skilled in the art unless otherwise further defined.

In the following, reference is made to embodiments of the invention. However, it should be understood that the invention is not limited to specific described embodiments. Instead, any combination of the following features and elements, whether related to different embodiments or not, is contemplated to implement and practice the invention. Furthermore, in various embodiments the invention provides numerous advantages over the prior art. However, although embodiments of the invention may achieve advantages over other possible solutions and/or over the prior art, whether or not a particular advantage is achieved by a given embodiment is not limiting of the invention. Thus, the following aspects, features, embodiments and advantages are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

A food product is susceptible to microbial contamination during virtually all steps of preparation. While refrigeration can retard microbial growth, such treatment does not necessarily kill bacteria. Accordingly, microorganisms can still survive through refrigeration, and worse, some microorganisms can still grow and produce harmful substances during refrigerated storage. It is desirable to sanitize equipment or devices and process foods using a combination of the cooling properties of cooling agents with the antimicrobial capability of antimicrobial agents. Embodiments of the invention generally provide exposing a target item to a treating agent, wherein the treating agent comprises an antimicrobial carrier chemical and a cooling agent.

As used herein, the term “antimicrobial” refers to a physical or chemical agent capable of causing greater than 90% reduction (1-log order reduction) in the population of bacteria or spores within 10 seconds at 60° C. The antimicrobial composition used in embodiments the invention preferably provides greater than a 99% reduction (2-log order reduction), and more preferably greater than a 99.99% (4-log order reduction), and most preferably a 99.999% (5-log order reduction) in such a population preferably within 60 seconds at 60° C, and more preferably within 10 seconds at 60° C.

As used herein, the phrase “target item” refers to equipment, devices, food products, pharmaceutical products, or other items that are in need of sanitation, preserving, or otherwise protecting from or treated for pathogenic microorganisms.

As used herein, the phrase “food or food product” generally refers to all types of foods, including, but not limited to, meats, including ground meats, poultry, seafood, produce including vegetables and fruit, dry pasta, breads and cereals, and fried, baked or other snack foods. The food may be in solid or liquid form, such as beverages or juices. The current inventive method may be used in conjunction with any food that is able to support microbial, fungal, bacterial, or viral growth, including unprocessed or processed foods.

One embodiment of the current invention provides a method of processing a target item by exposing the target item to a treating agent that contains an antimicrobial carrier chemical and a cooling agent. The treating agent is preferably in a solid form. The antimicrobial carrier chemical remains present in the treating agent while the treating agent is in its solid form. As heat is absorbed by the treating agent, the treating agent converts to a gas by sublimation, or melts into a liquid, and the antimicrobial carrier chemical is released from the treating agent. Once released, the antimicrobial carrier chemical contacts the target item, thus providing antimicrobial effects. Alternately, after the solid melts into a liquid, the liquid then evaporates, releasing the antimicrobial carrier chemical. In another alternative, the solid melts into a liquid that contains the antimicrobial carrier chemical and the mixture of the liquid and the antimicrobial chemical contacts the target item. Preferred treating agents contain at least 90% by weight cooling agent. Preferred treating agents also contain at least 0.1% by weight antimicrobial agent, preferably more than 1% by weight antimicrobial agent, and more preferably at least 5% by weight antimicrobial agent.

In some preferred embodiments, the treating agent does not contact said target item while in a solid form. The treating agent may be placed into a target item treatment area, package, or storage container adjacent to or in an adjoining compartment with the target item. The treating agent absorbs the heat from the target item, thus cooling the target item. Alternatively, the treating agent absorbs heat coming into the treatment area, package, or storage container, thus keeping the target item at desired temperature.

Preferred methods of processing a target item can be used to treat the target item while in any type of treatment device known to one of ordinary skill in the art. Examples for processing food products include a tunnel, tumbler, blender, plate, chamber, vessel, and combinations of these devices. Some preferred embodiments capture and recycle the cooling agent.

In one aspect of the current invention, a method of packaging a target item is provided. The method places a target item into a container and adds a treating agent that contains an antimicrobial carrier chemical and a cooling agent as described above to the container. The treating agent melts or sublimes to keep the interior of the container, and thus the target item, at a desired temperature while also contacting the contained target item with the antimicrobial carrier chemical. The container is typically, but not necessarily, a food storage container, or a food transportation container. In one embodiment, the food is packaged for sale or distribution with the treating agent placed in the package. The treating agent may be in direct contact with the target item, or may be separated from the food by packaging material, or in a separate compartment of the container.

A further aspect of the current invention provides a product that is a treating agent comprising a cooling agent and an antimicrobial carrier chemical agent. In one preferred embodiment, the cooling agent is in solid form. The antimicrobial agent is present in the cooling agent until the cooling agent melts or sublimes, releasing the antimicrobial agent. Melting or sublimation of the treating agent occurs as the treating agent absorbs heat from the target item or the surrounding environment. The treating agent preferably contains at least about 0.1 ppm by weight antimicrobial agent, and more preferably about 1 to 100 ppm by weight antimicrobial agent. The antimicrobial carrier chemical can be any antimicrobial agent known to one of ordinary skill in the art that provides the antimicrobial effect desired, acts a solvent of other additives and antimicrobials, and can be combined with a cooling agent. Preferred antimicrobial carrier chemicals include ethanol, polyethylene glycol, and terpenes. The cooling agent can be any suitable material for cooling target items. Preferred cooling agents include nitrogen (N₂), carbon dioxide (CO₂), and mixtures of N₂ and CO₂.

Liquid CO₂ is usually maintained at a temperature of about −60° C. at a pressure of 5.11 atm. In embodiments of the invention, the antimicrobial agents used may have freezing points higher, lower, or similar to that of liquid CO₂. Embodiments of the invention can involve mixing one or more antimicrobial agents with the carrier chemical to a final concentration without affecting the freezing point of the carrier chemical. In a preferred embodiment, a combined solution prepared using a carrier chemical and one or more antimicrobial agents should not have a freezing point higher than that of liquid CO₂. In other embodiments of the invention, it may be desirable to tune the freezing point of a mixture containing a carrier chemical and one or more antimicrobial agents. In one embodiment of the invention, liquid CO₂ combined with a carrier chemical/antimicrobial mixture is fed to an ice press to form dry ice. Yet another embodiment of the invention involves feeding liquid CO₂ and a carrier chemical/antimicrobial mixture to an ice press as separate streams, which then combine in the press to generate dry ice “snow” containing antimicrobial properties. In embodiments of the invention, the antimicrobial agents in the antimicrobial formulation are listed by the U.S. Food and Drug Administration as being GRAS (Generally Recognized as Safe).

The antimicrobial formulation can essentially contain an alcohol, a terpene, or polyethylene glycol as a carrier chemical in various embodiments. An alcohol is any organic compound in which a hydroxyl group (—OH) is bound to a carbon atom of an alkyl or substituted alkyl group. The general formula for a simple acyclic alcohol is CₙH₂ₙ₊₁OH. Food grade alcohol, ethanol, is a carrier chemical that has a very low freezing point, and can be used in one embodiment of the invention. Terpenes are another large group of chemicals compounds found in nature that act as effecting carrier chemicals with low freezing points. One such example is D-limonene, present in orange peel and extracted from the orange skin. The freezing point of D-Limonene is suitable for liquid CO₂ storage conditions. In general, ethanol and D-Limonene can be considered to be effective carrier chemicals used in formulation preparation with desired antimicrobial ingredients. Polyethylene glycol is a non-toxic liquid with low molecular weight, and is a common ingredient of antimicrobial pharmaceuticals.

Various food additives listed as GRAS can be dissolved directly into the carrier chemical and then mixed with liquid CO₂ or CO₂ in “snow” form before being extruded as pellets or blocks. Another embodiment of the invention can involve mixing one or more food additives with water, and then adding the solution to the carrier chemical to a final concentration without affecting the freezing point of the carrier chemical.

One embodiment of the invention involves the addition of the food additive MIRENAT-N, manufactured by Vedeqsa Lamirsa Group based in Barcelona, Spain and distributed in the U.S. by A & B Ingredients (Fairfield, N.J.). MIRENAT-N is manufactured from a naturally occurring antimicrobial compound, and its active ingredient is lauric arginate (N-lauroyl-L-Arginine ethyl ester monohydrochloride). The formulation available for sale contains about 10% active lauric arginate and 90% food grade propylene glycol. It is possible to substitute ethanol for propylene glycol as the carrier chemical when using MIRENAT-N. This antimicrobial was approved to be GRAS in September 2005 for use in meat and poultry products. Advantages of using MIRENAT-N include: minimal modification of original product, low application use dosage, and well known antimicrobial activity. Based on the manufacturer's specifications, MIRENAT-N can be manufactured to be lower than 11% active in ethanol. MIRENAT, either in propylene glycol, or ethanol, when treated with meat or poultry, can lose its efficacy over time, due to enzymatic reactions. Such problems can be overcome by adding other preservatives or antimicrobials to MIRENAT-N.

Other antimicrobial additives used in embodiments of the invention could include natural lactic acid, lactates, gluconates, and lacititol. The solubility of the following products manufactured by Purac (Lincolnshire, Ill.) was tested: potassium gluconate, ammonium lactate, potassium lactate, sodium lactate, sodium lactate powder, and sodium diacetate. Based on solubility testing, all liquid forms of these additives were found to be ethanol soluble. Other antimicrobial additives could include parabens, a group of chemicals which are derivatives of phenol. Parabens are widely used as preservatives in the cosmetic and pharmaceutical industries, and are also popular in the meat processing industry. Methyl paraben, sold by The KIC Group (Vancouver, Wash.), is also soluble in ethanol and not soluble in water. Thus, methyl paraben can be a preservative or antimicrobial added in one embodiment of the composition with ethanol as the carrier chemical.

Other antimicrobials that are not directly soluble in ethanol but soluble in water can be also be used in embodiments of the invention. Examples include potassium nitrite and potassium nitrate. These salts can be dissolved in water and further mixed with ethanol. The final composition of ethanol can be adjusted such that it does not freeze under liquid CO₂ storage conditions. The ethanol composition could be adjusted by starting with an amount of high purity ethanol and diluting the ethanol with water containing antimicrobials, such that a final composition is still compatible with liquid CO₂ temperatures.

In general, salts of organic acids (propinates, sorbates, benzoates and lactate) are preservatives that act by increasing the proton concentration of the cytoplasm of many microbes. Under mild conditions, they are protonated, since they are weak acids. The relative non-polarity of these salts allows the salts to penetrate the cellular membrane of bacteria and other microorganisms. Once inside the cell, these acids dissociate (releasing protons), due to the lower proton concentration of cytoplasm. Microorganisms, to maintain their proton concentration, they must compensate for these acids by discharging protons using ATP synthesis. This in turn disrupts ATP synthesis, and causes the microbes to die. Hence, the addition of these salts can enhance the antimicrobial efficacy of the composition proposed in the invention.

Other GRAS chemicals listed as food additives, but not antimicrobials, can also be introduced in the formulation to bring additional benefits other than antimicrobial effects. These additives include flavoring agents, flavor enhancers, intensifiers, emulsifiers, binders, fillers, gelling agents, plasticizers, stabilizers, suspending agents, whipping agents, sweetening agents, flavoring agents, colors, enzymes, antioxidants, sequestrants, wetting agents, surfactants, curing and pickling agents, firming agents, fumigants, humectants, leavening agents, processing aids, surface active agents, surface finishing agents, synergists, and texturizers.

The current invention will now be further described in terms of one embodiment of the current invention that uses solid CO₂ (dry ice) as the cooling agent and an antimicrobial carrier chemical such as D-limonene or ethanol. The dry ice product can be manufactured in the form of blocks, pellets, flakes, powders, and other possible forms containing carbon dioxide and terpene. The dry ice product is essentially void of water. What is meant by “essentially void of” is that the dry ice product, if it contains water, will comprise less than 5% by weight (wt. %) water. Typically, the water content will be less than 1 wt. %. Moisture levels of up to 5,000 ppm may be helpful in maintaining the desired shape of the product. The major constituent of the dry ice based treating agent is carbon dioxide. The alcohol or terpene concentration in the treating agent can vary widely and can depend upon the end use of the product and, in particular, the product being treated and the environment surrounding the treated product. Only minute amounts of alcohols or terpenes are required to contact the target item to provide an antimicrobial effect.

The exact form of the treating agent can vary and, accordingly, a wide variety of forms can be manufactured and used depending upon the target item to be treated and the purpose of such treatment, such as storage, transport, or commercial sale display of food products. For example, if the target item is stored in large rooms, blocks of dry ice ranging from 5 to 50 lbs. can be used. Likewise, if the target item to be stored, transported, or displayed for sale requires direct contact of the dry ice product, smaller manufactured shapes can be provided. Pellets of dry ice in the range of 1/16 inch to 1 inch can be formed. In addition, powders such as snow, flakes, or chips can be formed by methods known in the art.

In one embodiment of the invention, it has been found to be particularly useful to incorporate the antimicrobial carrier chemical into the carbon dioxide during the dry ice manufacturing process. The traditional first step in making dry ice is to manufacture carbon dioxide liquid. This is done by compressing CO₂ gas and removing any excess heat. The CO₂ is typically liquefied at pressures ranging from 200-300 pounds per square inch and at a temperature of −20° F. to 0° F., respectively. It is typically stored in a pressure vessel at lower than ambient temperature. The liquid pressure is then reduced below the triple point pressure of 69.9 psi by sending it through an expansion valve. This can be done in a single step or, in many cases, by reducing the liquid pressure to 100 psi at a temperature of −50° F. as a first step to allow easy recovery of the flash gases. The liquid CO₂ is expanded inside a dry ice manufacturing press to form a mixture of dry ice solid and cold gas. The cold gas is vented or recycled and the remaining dry ice snow is then compacted to form blocks. Dry ice is typically compacted to a density of approximately 90 lb/ft³.

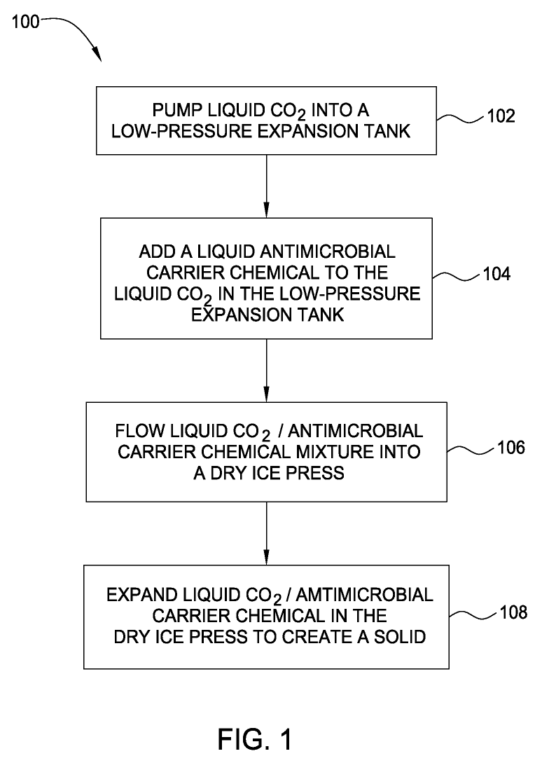

In general, to manufacture the dry ice treating agent, an antimicrobial carrier chemical is combined with liquid carbon dioxide at a pressure above the triple point of CO₂ (70 psi), allowing the antimicrobial to fully dissolve in the liquid CO₂. FIG. 1 is a flow diagram of a process 100 used to create a dry ice treating agent, according to one embodiment of the present invention. The process 100 includes a processing step 102 involving pumping liquid CO₂ into a low-pressure expansion tank and allowing the liquid CO₂ to expand to pressures above the triple point of carbon, typically to pressures of from about 70 psi to 100 psi. Processing step 104 involves adding a liquid microbial carrier chemical to the liquid CO₂ after the slight expansion of liquid CO₂ in the low-pressure expansion tank. The mixture of liquid CO₂ and liquid antimicrobial carrier chemical is then allowed to flow into a dry ice press in processing step 106. Finally, the liquid carbon dioxide/antimicrobial mixture is then expanded in the dry ice press to generate dry ice or “snow” in processing step 108. Alternatively, the antimicrobial composition can be introduced to CO₂ in “snow” form and further extruded into snow or any desired shape and form. This modified dry ice can then be collected or shaped such as by pressing or extrusion. In yet another embodiment, some amount of a liquid antimicrobial carrier chemical can be flashed together with gaseous CO₂ during pellet and block formation. The flashed gas, when recompressed to liquid CO₂, will inherently contain the antimicrobial carrier chemical in addition to any fresh liquid antimicrobial added during pellet and block formation. This scheme can be successfully adapted to existing dry ice plants.

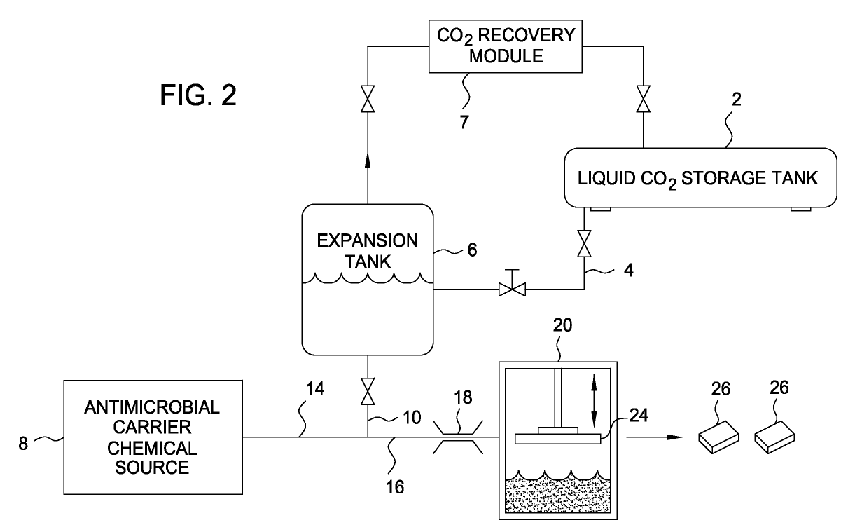

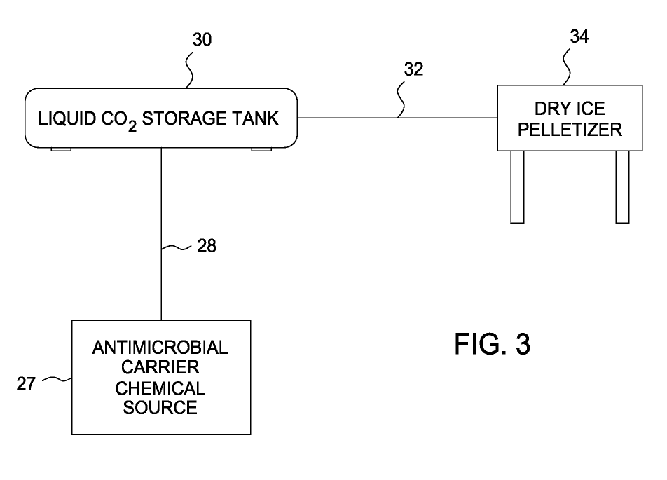

FIG. 2 and FIG. 3 depict representative apparatus and corresponding methods of forming a treating agent of dry ice with an antimicrobial carrier chemical according to the processing steps described in the process 100. Each figure represents a typical dry ice manufacturing apparatus and process in which FIG. 2 is a processing environment used to form blocks of dry ice, while FIG. 3 depicts a processing environment used to form dry ice pellets. These processing environments can be modified to incorporate an antimicrobial carrier chemical into the dry ice product. In FIG. 2, liquid carbon dioxide is stored in tank 2, typically at pressures of 200 to 300 psi. The liquid carbon dioxide from storage tank 2 is then passed via line 4 to a low-pressure expansion tank 6 wherein the liquid CO₂ is expanded to a pressure above the triple point of carbon dioxide (69.9 psi). Typically, the liquid CO₂ is expanded to pressures of from about 70 to 100 psi in expansion tank 6. What results is a mixture of gas and a dense, viscous carbon dioxide liquid. It is important that the liquid CO₂ is not formed into solid dry ice at this point because solids in the piping would disadvantageously reduce transport of the liquid. An antimicrobial carrier chemical from a vessel 8 is then injected into the liquid carbon dioxide. Injection of the antimicrobial carrier chemical can be done in the low-pressure expansion tank. As shown in FIG. 2, the antimicrobial carrier chemical from vessel 8 is fed via line 14 to mix with the liquid CO₂ from line 10. The mixture of antimicrobial carrier chemical and liquid CO₂ is passed via line 16 through an expansion orifice 18 into the dry ice press 20. Alternatively, although not shown, the mixture of antimicrobial and liquid CO₂ can be passed to a separate refrigeration unit, wherein the liquid CO₂ is frozen into a solid containing the entrapped antimicrobial carrier chemical.

As further shown in FIG. 2, the mixture of liquid CO₂ and antimicrobial carrier chemical is then allowed to expand inside the dry ice press 20. During expansion, the liquid CO₂ is converted to a solid form and the antimicrobial carrier chemical is trapped in the structural lattices of dry ice and/or by physical absorption during dry ice formation. Once the dry ice solid is formed, the solid particles can be compressed via platen 24 in press 20 into dry ice blocks 26.

The antimicrobial carrier chemical in the treating agent necessary for biological treatment is slowly released as the treating agent sublimes or melts during use. Higher concentrations and pressures of antimicrobial agents are preferred to achieve higher concentrations of antimicrobials in the treating agent. The preferred concentration of antimicrobial carrier chemical can vary depending upon the use of the treating agent and the target item treated.

FIG. 3 depicts a processing environment used to form dry ice pellets using a similar apparatus to that in FIG. 2. Liquid CO₂ is stored in tank 30, typically at pressures of 200 to 300 psi. The liquid carbon dioxide from storage tank 30 is then passed via line 32 directly to a dry ice pelletizer 34. Dry ice pelletizers are well known in the art. It is believed any dry ice pelletizer is capable of use with this invention. In the pelletizer, the liquid CO₂ is expanded to a pressure below 70 psi, resulting in a mixture of gas and carbon dioxide solid particles. An antimicrobial carrier chemical from the vessel 27 is fed via line 28 to mix with the CO₂ in the liquid CO₂ storage tank 30.

The liquid CO₂ is allowed to expand inside the dry ice pelletizer 34 and is converted to a solid form. While not bound by any theory of operation, if the antimicrobial carrier chemical is added during expansion, it is believed to be trapped in the structural lattices of dry ice. If the CO₂ is solid, either as particles or as extruded pellets during injection of liquid CO₂/antimicrobial mixture, the antimicrobial is believed to be contained in the dry ice by physical absorption. The solid CO₂ particles are extruded into pellets, typically ranging from 1/16 inch to 1 inch. As in the block dry ice, the antimicrobial carrier chemical in dry ice pellets necessary for biological treatment is slowly released as the carbon dioxide sublimes during use.

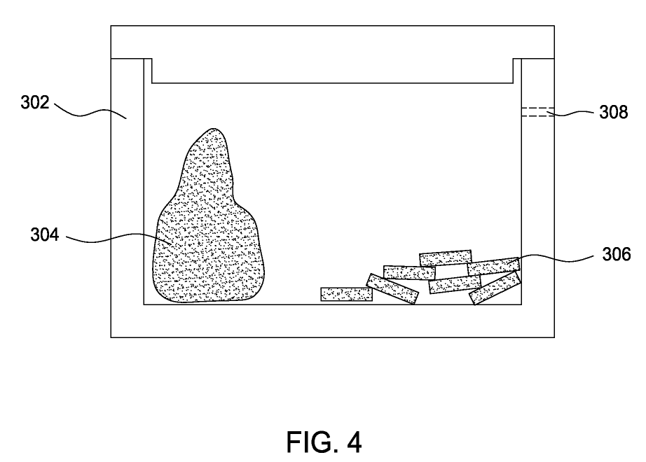

In one aspect of the current invention, a packaged product comprising a target item and a treating agent is provided. FIG. 4 depicts a method for placing a target item 304 into a package 302 and adding a solid treating agent 306 described above to the package. The cooling agent contained in the treating agent vaporizes or sublimes to cool or maintain the temperature in the package, while also exposing the target item and package interior to the antimicrobial agent. Some packages may be sealed, and thus may require a vent port 308 to vent the gases as the treating agent 306 sublimes. In one embodiment, the food is packaged for sale or distribution with the treating agent placed in the package. The treating agent may be in direct contact with the target item, or may be separated from the food by packaging material or a separate compartment of the package.

The treating agent of this invention improves the antimicrobial efficacy of cooling agents, such as dry ice, to better ensure safe target items, such as safe food products. The antimicrobial agent is effectively delivered into the cooling agent, such as dry ice, at a desired concentration such that during sublimation or melting of the cooling agent, the antimicrobial agent contacts the target item and exerts the desired antimicrobial effect for disinfection and/or sanitation purposes. The antimicrobial agent is released to disinfect target items, and to ensure significant reductions of pathogenic microorganisms. Because antimicrobial agents are often more stable under cold environments, the process provides the favorable conditions for antimicrobial agents to work at maximum reactivity. Since the release of the antimicrobial agent from the cooling agent is well regulated, target items receive antimicrobial agent slowly and constantly during the entire storage thereof, and accordingly, shelf life and quality of the target item is enhanced. Moreover, the cooling agent chills the target items efficiently, further providing benefits to target item. The cooling agent slows down the growth of pathogenic microorganisms, particularly pathogenic microorganisms that lead to spoilage in food, allowing food products to last longer and be safer. The cooling agent also slows down the enzymatic reactions in food, allowing the quality of food to be extended during storage. A cooling agent using dry ice sublimation also allows carbon dioxide to penetrate into microbial cells, lowering the intracellular pH of microbial cells, and causing those microbial cells to be more sensitive to the antimicrobial agent. Accordingly, a synergistic effect on antimicrobial efficacy can be achieved by combining a cooling agent, such as dry ice, and an antimicrobial carrier chemical, such as a terpene or alcohol.

Preferred processes and apparatus for practicing the present invention have been described. It will be understood and readily apparent to the skilled artisan that many changes and modifications may be made to the above-described embodiments without departing from the spirit and the scope of the present invention. The foregoing is illustrative only and that other embodiments of the integrated processes and apparatus may be employed without departing from the true scope of the invention defined in the following claims.

What Is Claimed Is:

1. A method of processing a target item, comprising:
a) exposing a target item to a treating agent, wherein the treating agent comprises an antimicrobial carrier chemical suspended in a cooling agent, the antimicrobial carrier chemical being in liquid or solid form; and
b) contacting the target item with the antimicrobial carrier chemical.

2. The method of claim 1, wherein the treating agent does not contact the target item while in the solid form.

3. The method of claim 1, wherein the treating agent comprises at least 90% by weight the cooling agent.

4. The method of claim 1, wherein the cooling agent is substantially anhydrous.

5. The method of claim 1, wherein the cooling agent is liquid carbon dioxide (CO₂).

6. The method of claim 1, wherein the antimicrobial carrier chemical is an alcohol.

7. The method of claim 6, wherein the alcohol is ethanol.

8. The method of claim 1, wherein the antimicrobial carrier chemical is propylene glycol.

9. The method of claim 1, wherein the antimicrobial carrier chemical is a terpene.

10. The method of claim 9, wherein the terpene is D-limonene.

11. The method of claim 1, wherein the antimicrobial carrier chemical contains one or more antimicrobial ingredients soluble in the antimicrobial carrier chemical.

12. The method of claim 11, wherein the one or more antimicrobial ingredients soluble in the antimicrobial carrier chemical is selected from the group consisting of lauric argenate, potassium gluconate, ammonium lactate, potassium lactate, sodium lactate, sodium diacetate, methyl paraben and combinations thereof.

13. The method of claim 1, wherein the antimicrobial carrier chemical contains one or more antimicrobial ingredients soluble in water.

14. The method of claim 13, wherein the one or more antimicrobial ingredients soluble in water is selected from the group consisting of potassium nitrite, potassium nitrate, and combinations thereof.

15. The method of claim 1, wherein the antimicrobial carrier chemical contains one or more additives that are not antimicrobials.

16. The method of claim 15, wherein the one or more additives that are not antimicrobials is selected from the group consisting of flavoring agents, flavor enhancers, intensifiers, emulsifiers, binders, fillers, gelling agents, plasticizers, stabilizers, suspending agents, whipping agents, sweetening agents, flavoring agents, colors, enzymes, antioxidants, sequestrants, wetting agents, surfactants, curing and pickling agents, firming agents, fumigants, humectants, leavening agents, processing aids, surface active agents, surface finishing agents, synergists, texturizers, and combinations thereof.

17. The method of claim 1, wherein the target item during the exposure step is in a treatment area selected from the group consisting of a tunnel, a tumbler, a blender, a plate, a chamber, a vessel, packages, transportation containers, and combinations thereof.

18. The method of claim 1, wherein the cooling agent is recycled.

19. A method of packaging a target item comprising:
a) placing a target item into a container;
b) adding a treating agent to the container, wherein the treating agent comprises an antimicrobial carrier chemical suspended in a cooling agent, the antimicrobial carrier chemical being in liquid or solid form; and
c) contacting the target item with the antimicrobial carrier chemical.

20. The method of claim 19, wherein the container is selected from a group consisting of a target item package, a food package, a food storage container, and a food transport container.

21. The method of claim 19, wherein the treating agent does not contact the target item while in the solid form.

22. The method of claim 19, wherein the treating agent comprises at least 90% by weight the cooling agent.

23. The method of claim 19, wherein the cooling agent is anhydrous.

24. The method of claim 19, wherein the cooling agent is liquid CO₂.

25. The method of claim 19, wherein the antimicrobial carrier chemical is an alcohol.

26. The method of claim 25, wherein the alcohol is ethanol.

27. The method of claim 19, wherein the antimicrobial carrier chemical is propylene glycol.

28. The method of claim 19, wherein the antimicrobial carrier chemical is a terpene.

29. The method of claim 28, wherein the terpene is D-limonene.

30. The method of claim 19, wherein the antimicrobial carrier chemical contains one or more antimicrobial ingredients soluble in the antimicrobial carrier chemical.

31. The method of claim 30, wherein the one or more antimicrobial ingredients soluble in the antimicrobial carrier chemical is selected from the group consisting of lauric argenate, potassium gluconate, ammonium lactate, potassium lactate, sodium lactate, sodium diacetate, methyl paraben and combinations thereof.

32. The method of claim 19, wherein the antimicrobial carrier chemical contains one or more antimicrobial ingredients soluble in water.

33. The method of claim 32, wherein the one or more antimicrobial ingredients soluble in water is selected from the group consisting of potassium nitrite, potassium nitrate, and combinations thereof.

34. The method of claim 19, wherein the antimicrobial carrier chemical contains one or more additives that are not antimicrobials.

35. The method of claim 34, wherein the one or more additives that are not antimicrobials is selected from the group consisting of flavoring agents, flavor enhancers, intensifiers, emulsifiers, binders, fillers, gelling agents, plasticizers, stabilizers, suspending agents, whipping agents, sweetening agents, flavoring agents, colors, enzymes, antioxidants, sequestrants, wetting agents, surfactants, curing and pickling agents, firming agents, fumigants, humectants, leavening agents, processing aids, surface active agents, surface finishing agents, synergists, texturizers, and combinations thereof.

36. A treating agent comprising an antimicrobial carrier chemical suspended in a cooling agent, wherein the antimicrobial carrier chemical is in liquid or solid form.

37. The treating agent of claim 36, wherein the antimicrobial carrier chemical is released when the cooling agent converts to a liquid or gaseous form.

38. The treating agent of claim 37, wherein the cooling agent converts a gaseous form as heat is absorbed by the cooling agent.

39. The treating agent of claim 36, wherein the antimicrobial carrier chemical is present in amounts of about 1 ppm to about 100 ppm.

40. The treating agent of claim 36, wherein the treating agent comprises at least 90% by weight the cooling agent.

41. The treating agent of claim 36, wherein the cooling agent is anhydrous.

42. The treating agent of claim 36, wherein the cooling agent is liquid CO₂.

43. The treating agent of claim 36, wherein the antimicrobial carrier chemical is an alcohol.

44. The treating agent of claim 43, wherein the alcohol is ethanol.

45. The treating agent of claim 36, wherein the antimicrobial carrier chemical is propylene glycol.

46. The treating agent of claim 36, wherein the antimicrobial carrier chemical is a terpene.

47. The treating agent of claim 46, wherein the terpene is D-limonene.

48. The treating agent of claim 36, wherein the antimicrobial carrier chemical contains one or more antimicrobial ingredients soluble in the antimicrobial carrier chemical.

49. The treating agent of claim 48, wherein the one or more antimicrobial ingredients soluble in the antimicrobial carrier chemical is selected from the group consisting of lauric argenate, potassium gluconate, ammonium lactate, potassium lactate, sodium lactate, sodium diacetate, methyl paraben and combinations thereof.

50. The treating agent of claim 36, wherein the antimicrobial carrier chemical contains one or more antimicrobial ingredients soluble in water.

51. The treating agent of claim 50, wherein the one or more antimicrobial ingredients soluble in water is selected from the group consisting of potassium nitrite, potassium nitrate, and combinations thereof.

52. The treating agent of claim 36, wherein the antimicrobial carrier chemical contains one or more additives that are not antimicrobials.

53. The treating agent of claim 52, wherein the one or more additives that are not antimicrobials is selected from the group consisting of flavoring agents, flavor enhancers, intensifiers, emulsifiers, binders, fillers, gelling agents, plasticizers, stabilizers, suspending agents, whipping agents, sweetening agents, flavoring agents, colors, enzymes, antioxidants, sequestrants, wetting agents, surfactants, curing and pickling agents, firming agents, fumigants, humectants, leavening agents, processing aids, surface active agents, surface finishing agents, synergists, texturizers, and combinations thereof.
